(19) United States
(12) Patent Application Publication
(10) Pub. No.: US 2026/0176014 A1
(43) Pub. Date: Jun. 25, 2026

(54) TRANSFER DEVICE FOR A CONTROLLED ENVIRONMENT

(71) Applicant: TT Innovation AG, Rotkreuz (CH)

(72) Inventors: Mike ZELLER, Wahlen (CH); Mathieu MÜLLER, Sierentz (FR); Robert LANDES, Bad Säckingen (DE)

(73) Assignee: TT Innovation AG, Rotkreuz (CH)

(21) Appl. No.: 19/424,553

(22) Filed: Dec. 18, 2025

(30) Foreign Application Priority Data
Dec. 19, 2024 (EP) 24221946.7

(51) Int. Cl. B65B 55/02; A61L 2/104; A61L 2/22; A61L 2/28; A61L 101/02; B65B 3/00

(52) U.S. Cl. CPC B65B 55/027; A61L 2/104; A61L 2/22; A61L 2/28; B65B 3/003; A61L 2101/02; A61L 2202/11; A61L 2202/14; A61L 2202/15

(57) ABSTRACT

A method for transferring objects (7) into a controlled environment (3) and a transfer device (1) having a controlled environment (3) having a passage (2) are provided. The passage (2) is closable using a cover (5) from the inside (4), in particular inside the controlled environment (3) and is closable using a package (6) containing an object (7) to be transferred from the outside (8), and having a decontamination device (9), which is configured for a decontamination of the passage (2). A checking unit (10) is configured for a check of a function of the decontamination device (9).

### CROSS-REFERENCE TO RELATED APPLICATIONS

This application claims priority from European Patent Application No. 24221946.7, filed Dec. 19, 2024, which is incorporated herein by reference as if fully set forth.

### TECHNICAL FIELD

The invention relates to a transfer device having a controlled environment comprising a passage, wherein the passage is closable using a cover from the inside, in particular within the controlled environment, and is closable from the outside using a package containing an object to be transferred, and having a decontamination device which is configured for a decontamination of the passage.

The invention furthermore relates to a method for transferring an object into a controlled environment, wherein the package is subjected to a decontamination before the object is brought into the controlled environment.

### SUMMARY

The object of the invention is to improve such transfer devices. The object of the invention is also to improve such methods.

According to the invention, one or more of the features disclosed herein are provided to achieve the object. In particular, to achieve the mentioned object, it is therefore provided in a transfer device of the type described at the outset that a checking unit be configured for checking at least one function of the decontamination device. In this way, for example, a fault or deficiency of the decontamination device or a decrease or a failure of the function of the decontamination device is preferably detectable automatically and/or promptly. The function of the decontamination device can be definable as a decontamination function. The transfer device can be configured to transfer objects, for example medical containers, in particular vials, syringes, carpules (cylindrical ampoules), into the controlled environment. Objects can also comprise further objects such as emergency gloves, tweezers, consumable material, tubing, stoppers, syringes, and/or bags. The transfer device can also be a rapid transfer port (RTP). The package of the object can close the passage, through which the object can be transferred into the controlled environment, from the outside in this case. The outside can be defined here, for example, as an environment outside the controlled environment. The outside can be, for example, a less controlled environment than the controlled environment in this case. However, the outside can also be, for example, a second controlled environment. The cover can close the passage from the inside, thus from inside the controlled environment. The package can be a so-called tub, which forms a receptacle container for medical and/or pharmaceutical containers. The package can comprise a cover made of a nonwoven fabric in this case, such as Tyvek. The package can be held in an overpack before it is supplied to the passage. This overpack can be removed before the decontamination. A seal can be seated in the passage, which together with the package and/or with the pressed-on cover on the passage creates a sealing function, so that air exchange cannot take place between the controlled environment and outside the controlled environment. The package can be pressed from the outside using a handling device against the passage, wherein a sealing function can thus be achieved using the package and the controlled environment. Alternatively, the package can be applied to the passage, in particular the seal located therein, by a clamping mechanism, clamping in a manual or automated manner. A sealing function can also be achievable, for example, in that the package is held from outside the controlled environment on the passage so that a gap is present between the package and the controlled environment. Air from inside the controlled environment can flow to the outside of the controlled environment through this gap, so that contaminants cannot penetrate into the controlled environment. Therefore, a direct contact does not have to be present between the package and the controlled environment for a sealing function.

The controlled environment can be characterizable, for example, as a bounded spatial area in which defined environmental conditions, in particular with respect to an air purity and/or a surface cleanliness, can be created and/or maintained, for example by a control of an air exchange with an outside world of the controlled environment. The control of the air exchange can be implemented, for example, by a complete exclusion of the air exchange in normal operation (for example except for a closed air circulation) or a consistent specification of a direction of the air exchange (into the controlled environment or out of the controlled environment). Examples of controlled environments comprise isolators, restricted access barrier systems (in particular of the open or closed type), containments, and glove boxes.

Therefore, various processes for processing greatly varying products, such as pharmaceutical products, can take place within the controlled environment, in particular the containment or the isolator, wherein the risk of a contamination of at least one product by particles and/or contaminants from outside the controlled environment can be decimated. Such pharmaceutical products can each comprise, for example, a pharmaceutical container for accommodating a pharmaceutical preparation. A list of examples of such pharmaceutical containers comprises at least vials, syringes, carpules, and other fillable pharmaceutical containers.

The passage can be understood, for example, as the volume which is formed by the passage and the adjacent surfaces, in particular cover and package.

The controlled environment can be characterizable, for example, as a volume which is enclosed when the passage is closed. Opening the passage would not enlarge the controlled environment in this understanding, although set environmental conditions would then extend to the passage. Rather, in this sense the controlled environment would be connected to a further space for leveling of environmental conditions by opening the passage.

In one advantageous embodiment, it can be provided that the decontamination device comprises an emission unit. In this way, for example, the decontamination device can emit substances or radiation in order to decontaminate one or more objects therewith. In particular, the decontamination device can comprise a radiation generator. In this way, for example, objects to be decontaminated can be decontaminated using radiation. Inter alia, thermal radiation, high-energy particle radiation, such as e-beam, gamma radiation, and/or microwave radiation can be used as the radiation. A pulsed light or a plasma can also be provided for the decontamination. The radiation generator preferably comprises a UVC lamp, by which objects can be decontaminated. Alternatively or additionally, the decontamination device can comprise a decontaminating agent distribution device, using which, for example, substances can be distributed in order to thus decontaminate objects. The decontaminating agent distribution device can be configured, for example, for nebulizing, spraying, injecting, jetting, or vaporizing. In particular, the decontaminating agent distribution device can be an H₂O₂ nebulizer. In this way, for example, objects can be decontaminated using H₂O₂. Nebulizing can be defined as spraying and/or vaporizing substances. In addition to H₂O₂ (hydrogen peroxide), other germicidal chemicals can also be usable.

In one advantageous design, it can be provided that the decontamination device is arranged in the cover. In this way, for example, the cover, which can comprise the decontamination device, can close the passage and during this carry out the decontamination of the package located in the passage. The decontamination device can be integrated here into the cover. Alternatively or additionally, the decontamination device can be arranged in the passage. In this way, for example, the passage and/or the package located in the passage can be decontaminated by the decontamination device arranged in the passage. It can additionally be advantageous that the passage and/or the package can be decontaminated by both decontamination devices, that in the passage and that in the cover. This can increase the effectiveness and rapidity of the decontamination. The decontamination device can be integrated into the passage.

After the decontamination with respect to time, the container held in the package can be transferred into the controlled environment. The transferred containers or other transferred objects can be further processed in the controlled environment at processing stations, such as filling stations or closing stations.

In one advantageous design, it can be provided that the checking unit is arranged inside the passage. In this way, the checking unit can be arranged spatially close to the decontamination and can check, for example, the function of the decontamination device there, namely in the passage, where the decontamination takes place. In addition, this arrangement can be space-saving and efficient. Alternatively or additionally, it can be provided that the checking unit is arranged outside the passage, for example is integrated into the cover. In this way, for example, the passage can be constructed less complexly if the checking unit is arranged outside the passage.

In one advantageous design, it can be provided that the checking unit comprises at least one sensor. The at least one sensor can be configured, for example, for measuring process parameters, using which a function of the decontamination device can be determined. In particular, the at least one sensor can be arranged in the decontamination device. In this way, for example, the function of the decontamination device is checkable using the sensor during or immediately after the decontamination. Likewise, it can also be advantageous that an additional setup location for an external sensor is not necessary. Moving the decontamination device to an external sensor can be superfluous. In particular, the at least one sensor can be integrated into the decontamination device. Alternatively or additionally, the at least one sensor can be arranged outside the decontamination device. Such an arrangement can provide structural advantages, since, for example, more space can be available for the emission unit in the decontamination device. It can likewise thus be advantageous that the decontamination is not obstructed. The decontamination can also be completely detectable by the sensor arranged outside the decontamination device. Furthermore, it can be advantageous that a sensor arranged outside the decontamination device can be larger than a sensor inside the decontamination device.

The sensor can also be, for example, a volume flow sensor which can measure how much H₂O₂ was introduced.

In one advantageous design, it can be provided that the at least one sensor is a testing sensor, using which the decontamination device is testable. Therefore, for example, the function of the decontamination device can be tested. In particular, the at least one sensor can have a testing field using which the decontamination device is testable. The testing field can therefore test, for example, the function of the decontamination device in that the decontamination device is held on the testing field. The testing field can be spanned between multiple sensors for checking the decontamination device. Alternatively or additionally, it can be provided that the at least one sensor has an indication sensor which triggers a check. In this way, for example, multiple sensors can be present, wherein one sensor has an indication sensor which can trigger a check of the decontamination device. Process parameters can therefore advantageously be detected which are based on previously determined validated parameters, wherein the process parameters ensure a basis for a reoccurring decontamination.

In one advantageous design, it can be provided that at least one point of the decontamination device, preferably having the potentially least decontamination effect, is checkable using the at least one sensor. The decontamination device cannot decontaminate uniformly well at every point, for example, but rather points can be present where the decontamination device can decontaminate less effectively. It can therefore be advantageous to measure this point of the potentially least decontamination effect using the at least one sensor, since it can thus be established whether the point of the potentially least decontamination effect is still above a predetermined parameter for the function of the decontamination.

In one advantageous design, it can be provided that the at least one sensor is configured to detect a quantity of a decontaminating agent emitted by the decontamination device. In this way, the sensor can measure, for example, a measured value of a measured variable, with which inferences about the function of the decontamination device can be possible.

In one advantageous design, it can be provided that a detection area of the checking unit has a surface equal in size to the passage. In this way, for example, a decontamination device can be checked which has a surface that corresponds to the surface of the passage. The function can therefore be monitored over the entire area of the passage. Alternatively, it can be provided that a detection area has a larger surface than the passage. In this way it can be ensured, for example, that the checking unit can completely cover the decontamination device, wherein an additional border area of the decontamination device can also be covered. The detection area can be spanned here by individual measurement points. The surface of the passage can be a plane of an opening through which the objects are transferable into the controlled environment.

In one advantageous design, it can be provided that a detection area of the checking unit is larger than an emission area of the decontamination device. It can therefore be ensured, for example, that the checking unit can detect the entire emission area of the decontamination device. In particular, it can be provided that so-called worst-case positions are covered. Worst-case positions can be positions at which the decontamination device provides a lower decontamination performance, for example in corners of the decontamination device.

In one advantageous design, it can be provided that the checking unit comprises a checking means sensitive for the decontamination on the package. In this way, for example, the checking unit can be part of the package which closes the passage from the outside. During the decontamination, the package is decontaminated, wherein, for example, it can then be established directly on the package whether the decontamination device functions as specified. In particular, the checking means can be a consumable material. Therefore, the checking means can be disposed of, for example, after the checking of the decontamination device. An evaluation of the checking means can take place optically, for example.

In one advantageous design, it can be provided that a checking unit is arranged in the decontamination device. In this way, for example, the checking unit can be formed in one piece with the decontamination device. It can likewise be advantageous that the checking unit can check the decontamination device during each decontamination procedure. The checking unit can be integrated into the decontamination device.

In one advantageous design, it can be provided that multiple sensors are assigned to individual decontamination elements. Decontamination elements can be, for example, individual UVC lamps or individual decontaminating agent distribution devices. The sensors can be arranged, for example, between UVC lamps.

In one advantageous design, it can be provided that the checking unit and the decontamination device are movable in relation to one another. In this way, for example, the decontamination device can be moved toward the checking unit or vice versa. In particular, it can be provided that the checking unit and the decontamination device are movable in relation to one another for a change between a checking position and an operating position. A checking position can be the position in which the decontamination device is checked. An operating position can be the position in which the decontamination device decontaminates the passage. In this way, for example, in the checking position the checking unit and the decontamination device can be moved toward one another, wherein in an operating position the decontamination device and the checking unit can be spaced apart from one another. If the checking unit is integrated into the decontamination device, checking position and operating position can be identical. Alternatively or additionally, it can be provided that the checking unit and the decontamination device are movable in relation to one another such that the decontamination device can be brought into or out of a detection area of the checking unit. In this way, for example, the decontamination device can be moved into or out of the detection area of the checking unit and is checkable there. Preferably, at least the checking unit may not be permanently installed, so that the decontamination device can move out of the operating position into the checking position toward the checking unit and can move from the checking position into the operating position away from the checking unit.

In one advantageous design, it can be provided that the decontamination device is at least temporarily movable into the controlled environment. In this way, for example, the decontamination device can be brought into an environment of greater cleanliness and/or having higher pressure level. Alternatively or additionally, it can be provided that the checking unit is arranged in the controlled environment. In this way, the checking unit can be arranged, for example, in the controlled environment having greater cleanliness and/or higher pressure level.

In one advantageous design, it can be provided that the transfer device comprises a comparison unit, which is configured to compare a quantity measured by the at least one sensor with a parameter. In this way, for example, an inference about the function of the decontamination device is possible. In particular, the parameter can be a value range and/or a setpoint value and/or a minimum value and/or a limiting value. The function of the decontamination device can result from the mentioned parameters. For example, a measurement of the input power of a UVC lamp can be used as an indicator of an operating state.

In one advantageous design, it can be provided that the or a comparison unit is configured for a comparison of a measurement result of at least one sensor, which is integrated into the decontamination device, with a measurement result of at least one sensor which is arranged outside the decontamination device. In this way, for example, the two measured values measured at the two sensors can be compared in order to derive the function of the decontamination device.

In addition, the features directed to a method are provided according to the invention for achieving the object mentioned at the outset. In particular, it is therefore provided in the case of the method to achieve the object mentioned at the outset that a function of a decontamination is checked. A function check of the decontamination can be carried out, for example, by checking a decontamination device which performs the decontamination. In this way it can be ensured, for example, that the decontamination runs effectively and safely. For example, a failure or a drop of a performance of the decontamination can be noticed early by testing the decontamination device. The method for transferring can in particular be carried out using the above-describe transfer device.

In one preferred application, it can be provided that the decontamination device is presented to a testing sensor. In this way, for example, the testing sensor can test the decontamination device and measure a measured value of a measured variable from which the function of the decontamination device results. The decontamination device can be presented to the testing sensor, for example, at time intervals. Therefore, for example, the decontamination device can be tested according to a previously defined plan and is therefore independent of a need-controlled test. Alternatively or additionally, the decontamination device can be presented to the testing sensor after achieving a predetermined criterion. In this way, for example, the decontamination device can be tested as soon as a drop or a change of the function of the decontamination device is measured. If a minimum value is not reached in a specific time, it can be provided that the passage is not released for following process steps, in particular is not released for a handling unit, but rather a renewed decontamination is carried out.

In one preferred application, it can be provided that the emission unit is presented after a release of an access to an external sensor to determine a radiation dose. In this way, for example, the emission unit of the decontamination device can be guided for checking to the external sensor, which can then measure a radiation dose of the emission unit. This radiation dose can therefore be measured with respect to the decontamination. Alternatively or additionally, the emission unit can be presented to the external sensor for determining the radiation dose before closing the access. In this way, for example, the emission unit can be checked before the decontamination. Alternatively or additionally, the emission unit can be presented between two decontamination cycles of the controlled environment to the external sensor to determine the radiation dose. In this way, for example, a check of the decontamination between the two decontamination cycles can be achievable, which advantageously does not interrupt operation in the controlled environment.

In one preferred application, it can be provided that a quantity measured by the testing sensor is compared with a parameter. In this way, for example, an inference can be possible about the function of the decontamination device. Alternatively or additionally, a quantity measured by the indication sensor can be compared with a parameter. In this way, for example, a check of the decontamination device can be triggered. Alternatively or additionally, it can be provided that the parameter is a value range and/or a setpoint value and/or a minimum value and/or limiting value. The function of the decontamination device can result from the mentioned parameters.

In one preferred application, it can be provided that the comparison unit outputs an alarm in the event of a deviation of the measured quantity from the parameter. The alarm can be, for example, an optical and/or acoustic notification. Alternatively or additionally, it can be provided that the deviation is noted in a memory. In this way, for example, a log file can be created and stored about when and with which values (quantity) the deviation has taken place.

In one preferred application, it can be provided that the package is subjected to a decontaminating agent and is then opened by a handling device. In this way, the decontamination of the package can be achievable. Likewise, the package can be opened in this way to remove objects, which are held in the interior of the package, in the controlled environment. The handling device can be, for example, a robot, which cuts open the package, in particular the cover of the package, using a cutting device, such as a blade. The package can remain here outside the controlled environment, for example.

In one preferred application, it can be provided that the decontamination device is presented at least temporarily to the checking unit during the opening of the package. In this way, for example, the function of the decontamination device can be tested at the same time as the work step of opening, which enables efficient work in the controlled environment. Moreover, it can therefore be established with a high probability, for example, whether the previously performed decontamination of the decontamination device was effective. Alternatively or additionally, it can be provided that the decontamination device is presented at least temporarily to the checking unit during the removal of contents of the package. In this way, for example, efficient work in the controlled environment can be possible. The decontamination device can preferably be presented by means of the handling device to the checking unit. The decontamination device can thus be arranged, for example, on a handling device or held or grasped by a handling device in order to be presented to the checking unit. Presenting can mean in this case that the decontamination device is guided to the checking unit and “shown” or brought into a detection area of the sensors of the checking unit, so that the check can be carried out.

In one preferred application, it can be provided that a measurement result of an, in particular the above-described, at least one sensor, which is arranged in the decontamination device, is compared with a measurement result of at least one sensor which is arranged outside the decontamination device. In this way, for example, the measured values measured at both sensors, that in the decontamination device and that outside, can be compared, wherein in the event of a deviation, a notification of a malfunction of the decontamination device or the sensors can be present. This can increase the security of the decontamination device and therefore of a transfer process.

In one preferred design, it can be provided that the passage is closed from the outside using a checking unit, wherein the checking unit comprises sensors. The checking unit represents a mockup of a package in this case. The decontamination device can advantageously be checked in a testing situation which corresponds to the operating position in this way.

### BRIEF DESCRIPTION OF THE DRAWINGS

The invention will now be described in more detail on the basis of several exemplary embodiments, but is not restricted to the exemplary embodiments. Further exemplary embodiments result by combination of the features of individual or multiple claims with one another and/or with individual or multiple features of the exemplary embodiments.

In the figures:

FIG. 1 shows a transfer device in a lateral sectional view in an operating position,

FIG. 2 shows the transfer device from FIG. 1 in a lateral sectional view in an operating position,

FIG. 3 shows the transfer device from FIGS. 1-2 in a lateral sectional view in a checking position,

FIG. 4 shows the transfer device from FIGS. 1-3 in a top view in an operating position,

FIG. 5 shows the transfer device from FIGS. 1-4 in a top view in a checking position,

FIG. 6 shows the transfer device from FIGS. 1-5 in a lateral sectional view, wherein sensors are arranged in a decontamination device,

FIG. 7 shows a transfer device in a lateral sectional view, wherein the decontamination device is arranged in a passage,

FIG. 8 shows a detail view of the passage from FIG. 2,

FIG. 9 shows a transfer device in a lateral sectional view with a checking package,

FIG. 10 shows a package with containers in the interior,

FIG. 11 shows a further package with containers in the interior,

FIG. 12 shows the package from FIG. 11 in an open state, and

FIG. 13 shows a view of a cover lower side and a UVC lamp.

### DETAILED DESCRIPTION

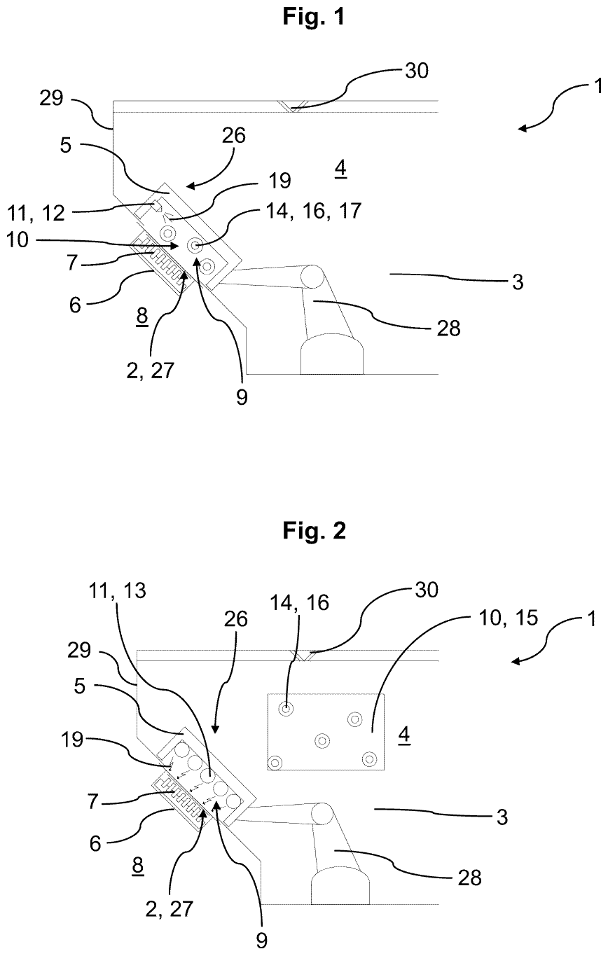

FIG. 1 shows the transfer device 1 designated as a whole by 1. The transfer device 1 comprises a controlled environment 3 having a passage 2. The passage 2 is closed from the inside 4 using a cover 5. The passage 2 is closed from the outside 8 using a package 6, which contains an object 7 to be transferred. The transfer device 1 comprises a decontamination device 9, which is configured to decontaminate the passage 2, and the surfaces terminating the passage. The controlled environment shown in FIGS. 1-7 and 9 is an isolator 29 which is shown open toward the right side of the image. The representations are used for illustration, wherein the isolator 29 is obviously not laterally open. It is also insofar clear that the inside area 4 of the isolator 29 is separated from the outside area 8.

FIG. 2 shows the transfer device 1, which comprises a checking unit 10 for checking a function of the decontamination device 9. The decontamination device 9 comprises an emission unit 11, which is designed in the exemplary embodiment of FIG. 1 as an H₂O₂ nebulizer 12 and in the exemplary embodiment of FIG. 2 as a UVC lamp 13. The H₂O₂ nebulizer 12 can distribute H₂O₂ in the passage 2 and therefore decontaminate the passage 2. The UVC lamp 13 can decontaminate the passage 2 by means of UVC radiation. In both decontamination methods, microbiological contaminants can be removed and/or killed.

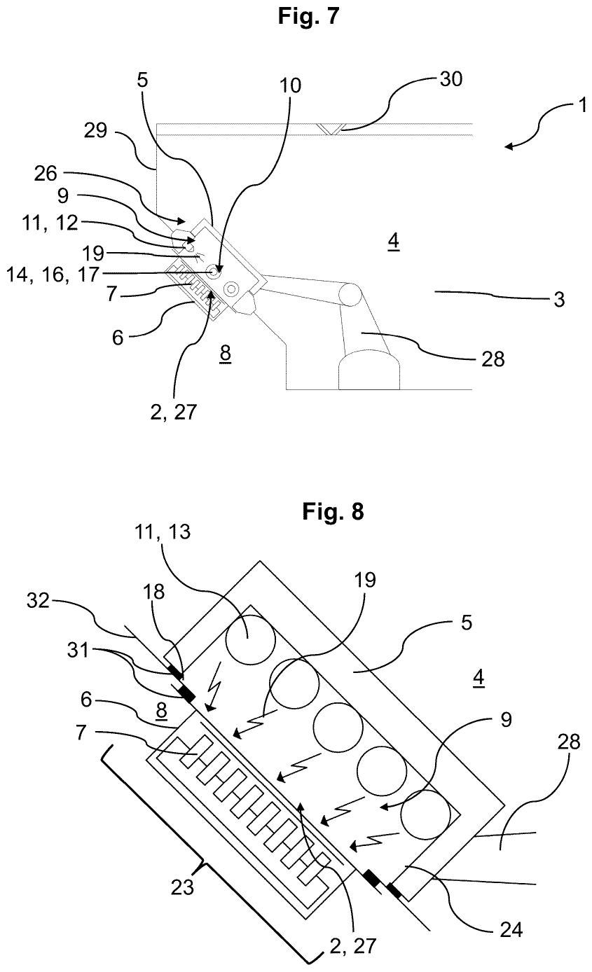

In the exemplary embodiments present in FIGS. 1, 2, 3, 6, and 9, the decontamination device 9 is shown in the cover 5. In the exemplary embodiment shown in FIG. 7, the decontamination device 9 is arranged in the passage 2.

In the exemplary embodiments shown in FIGS. 1, 2, 3, 4, 5, 6, and 9, the checking unit 10 is arranged outside the passage 2. In the exemplary embodiment shown in FIG. 7, the checking unit 10 is arranged inside the passage 2.

The checking unit 10 comprises multiple sensors 14, wherein sensors 14 are arranged in the decontamination device 9 in FIG. 1. In the transfer device 1 shown in FIGS. 2, 3, 4, and 5, the sensors 14 are arranged outside the decontamination device 9. The sensors 14 are arranged there in the controlled environment 3, wherein the sensors 14 comprise testing sensors 16 arranged in a testing field 15, using which the decontamination device 9 is testable.

The sensors 14 likewise comprise indication sensors 17 that can trigger a check of the decontamination device 9. The indication sensors 17 are integrated into the decontamination device 9, which is arranged in the cover 5, in the exemplary embodiment of FIG. 6.

The sensors 14 can check a point 18 of the decontamination device 9 having the potentially least decontamination effect.

The sensors 14 are configured to detect a quantity of a decontaminating agent 19 emitted by the decontamination device 9.

The checking unit 10 has a detection area 20 which has a larger surface 21 than a surface 22 of the passage 2.

The detection area 20 of the checking unit 10 is larger than an irradiation area 23 of the decontamination device 9. The checking unit 10 can therefore detect so-called worst-case positions 24 of the decontamination device 9.

In one exemplary embodiment (not shown), the package 6 comprises a checking means sensitive to the decontamination. This can be a label or an imprint, for example, which changes in an optically detectable manner under the effect of the decontaminating agent.

The checking unit 10 is arranged in the decontamination device 9 in the exemplary embodiment shown in FIGS. 1 and 6.

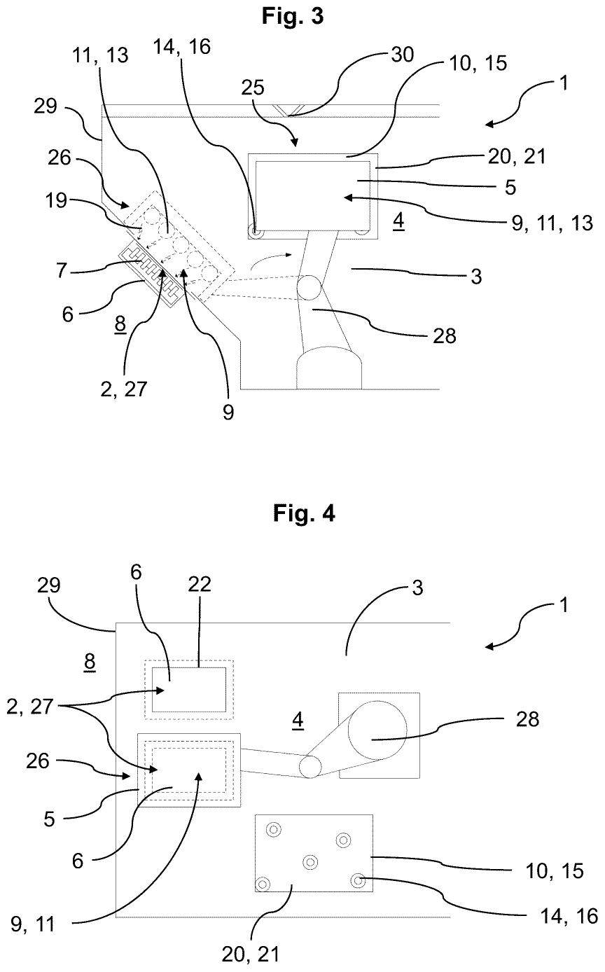

FIG. 3 shows how the checking unit 10 and the decontamination device 9 are movable in relation to one another to be able to change from a checking position 25 into an operating position 26, and vice versa. The decontamination device 9 can thus be brought into or out of a detection area 20 of the checking unit 10. In FIG. 3, the decontamination device 9 moves from the operating position 26 into the checking position 25.

The decontamination device 9 is temporarily movable into the controlled environment 3. The checking unit 10 is arranged in the controlled environment 3.

In one exemplary embodiment (not shown), the transfer device 1 comprises a comparison unit, which is configured to compare a quantity measured using a sensor 15 with a parameter. The parameter can be a value range and/or setpoint value and/or minimum value and/or limiting value.

The comparison unit is configured for a comparison of a measurement result of the sensors 14 which are integrated in the decontamination device 9 and a measurement result of a sensor 14 outside the decontamination device 9.

To transfer an object 7 into the controlled environment 3, the package 6 of the object 7 is subjected to a decontamination before the object 7 is brought into the controlled environment 3, wherein a function of the decontamination is checked. The function in this case means functioning of the decontamination device 9 as specified, so that the package 6 can be decontaminated. The package 6 of the object 7 is decontaminated using the decontamination device 9 before the object 7 is transferred into the controlled environment 3. Before, during, or after the transfer, a function check of the decontamination device 9 can be carried out here using the checking unit 10. The decontamination device 9 is presented to the testing sensor 16. This can take place at time intervals and/or after achieving a predetermined criterion. For example, a predetermined criterion can be a measured value of the indication sensor 17, by which the check is triggered.

The emission unit 11 is presented to an external sensor 14 to determine a radiation dose after releasing an access 27 and/or before closing the access 27 and/or between two decontamination cycles of the controlled environment 3 or after a period of time defined by the facility operator.

A quantity measured by the testing sensor 16 and/or indication sensor 17 is compared with a parameter. The parameter is a value range and/or a setpoint value and/or a minimum value and/or a limiting value.

The comparison unit outputs an alarm in the event of a deviation of the measured quantity from the parameter. Likewise, the comparison unit notes the deviation in a memory and/or log. A comparison takes place between a measurement result that is measured by the sensor 14, which is arranged in the decontamination device 9, and a measurement result that is measured by a sensor 14, which is arranged outside the decontamination device 9.

For the decontamination of the package 6, the package 6, or at least parts thereof, is subjected to a decontaminating agent 19 and subsequently opened by a handling unit. It can be subjected by the discharge of the decontaminating agent.

The decontamination device 9 can be temporarily presented to the checking unit 10, if necessary, during the opening of the package 6 and/or the removal of contents of the package 6 by means of the handling device 28. The decontamination device 9 is arranged on the handling device 28 and can be moved therewith.

The controlled environment 3 is an isolator 29 in the present exemplary embodiment. The controlled environment 3 has a laminar airflow 30, which preferably flows along a direction of the force of gravity and/or corresponds to a blowing-out direction.

FIG. 4 shows a controlled environment 3 having two passages 2, wherein a first passage 2 is presently being decontaminated by the decontamination device 9. The second passage 2 is closed from the outside 8 by the package 6. While the first passage 2 having a package 6 pressed thereon from the outside 8 is being decontaminated, the package 6 which is held in the second passage 2 can be opened by a handling device (not shown), for example cut open or stamped.

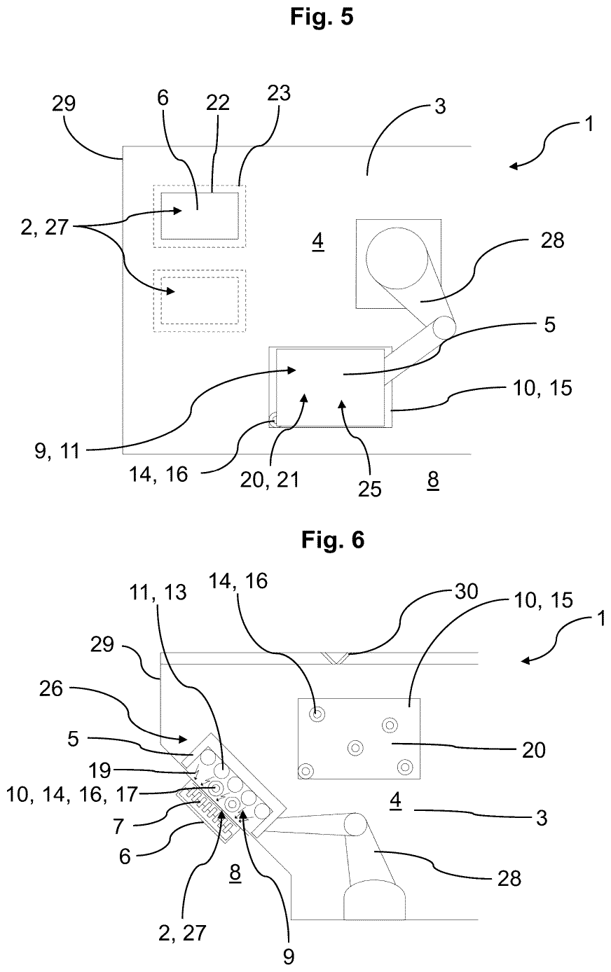

FIG. 5 shows the checking position 25, wherein the decontamination device 9 mounted on the handling device 28 is moved to the checking unit 10 and is tested there by the sensors 14, which are testing sensors 16, of the testing field 15.

The decontamination device 9 shown in FIG. 6 also comprises, in addition to the emission unit 11, sensors 14 which are designed as indication sensors 17 and testing sensors 16. In one exemplary embodiment (not shown), the sensors 14 of the emission unit 11 are designed as indication sensors 17. In another exemplary embodiment (not shown), the sensors 14 of the emission unit 11 are designed as testing sensors 15.

FIG. 8 shows a detail view of the passage 2 of the transfer device 1 from FIG. 2. In the detail view, seals 31 are indicated between the package 6 and a housing wall 32 of the controlled environment 3 and between the cover 5 and the housing wall 32, which indicate that air exchange cannot take place between the outside 8 and the inside 4 of the controlled environment 3. In a form which is not shown, a one-piece seal is arranged, preferably in tapering form, to prevent a shadow. The seals 31 are also present in the other figures, but are not shown. FIG. 8 also shows by way of example a point 18 where the decontamination device 9 potentially has the least decontamination effect. A worst-case position 24 at the border of the decontamination device 24 is shown by way of example in the decontamination device 9. The decontamination device 9 can provide a lower decontamination performance here than in the center of the decontamination device 9. The irradiation area 23 is smaller than the detection area 20 of the checking unit 10 (see FIG. 5).

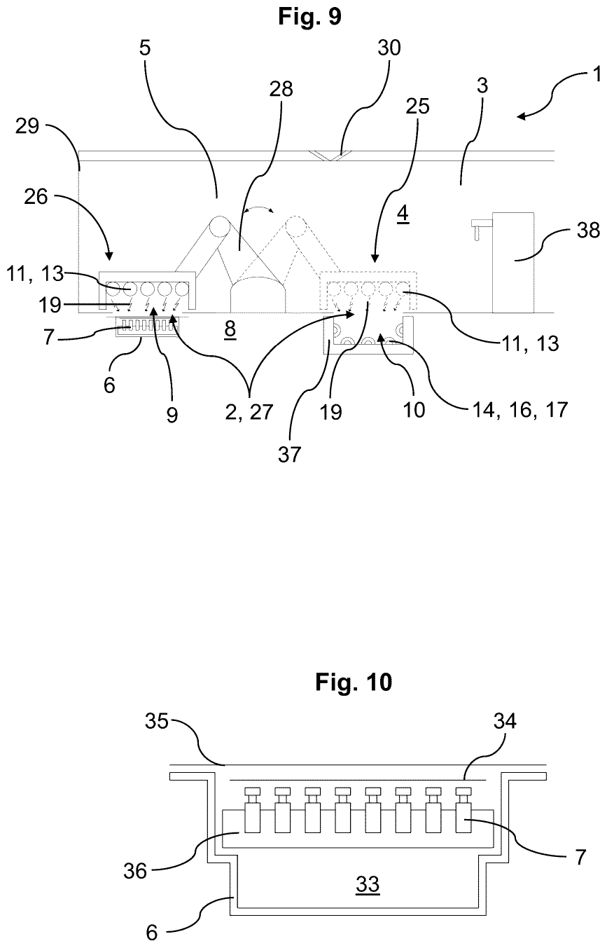

FIG. 9 shows a transfer device 1 of a controlled environment 3. To check the function of the decontamination device 9, a checking unit 10 in the form of a checking package 37 is held from the outside 8 on the passage 2. In a checking position 25, the handling device 28 moves the decontamination device 9 to the passage 2. During the checking of the decontamination device 9, the emission unit 11 operates as it would operate in the operating position 26 with a package 6. Sensors 14 are arranged in the checking package 37, wherein the sensors 14 are testing sensors. The function of the decontamination device 9 on the package 6 can advantageously be simulated with the aid of the checking package 37. A processing station in the form of a filling device 38 is arranged in the controlled environment 3 shown.

FIG. 10 shows the package 6 in a lateral sectional view, wherein the package 6 contains a container receptacle 36 having objects 7. The objects 7 are containers for filling or further processing, for example of medical products. The objects 7 are covered by an inside cover 34. The package 6 comprises a cover 35, wherein this cover 35 is subjected to the decontamination. An interior 33 of the package 6 is aseptic. The cover 35 is connected to the package 6 to form a seal.

FIG. 11 shows a further design of the package 6 in a lateral sectional view, wherein the package 6 contains a container receptacle 36 having objects 7.

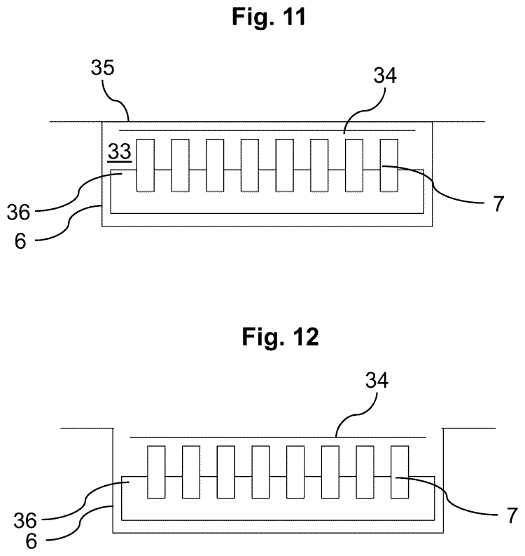

FIG. 12 shows the package 6 without the cover 35.

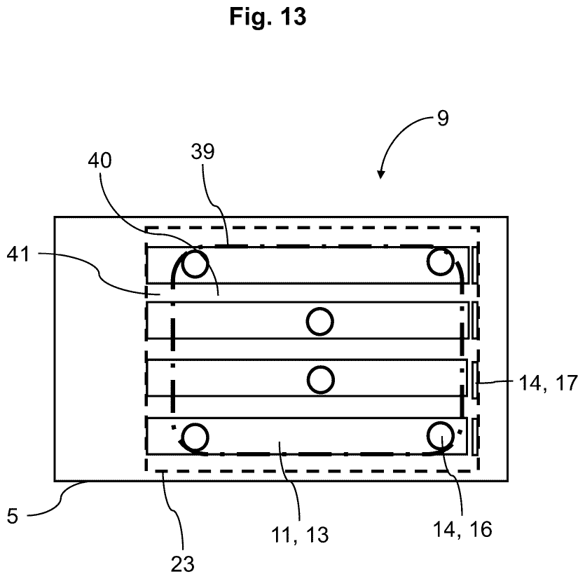

FIG. 13 shows a view of a lower side of the cover 5, wherein the decontamination device 9 is arranged in the cover 5 and comprises fluorescent tubes of a UVC lamp 13. The decontamination device 9 also comprises sensors 14, which are designed as testing sensors 16 and indication sensors 17. The dot-dash line corresponds to a circumference 39 of the passage 2 on which the cover 5 is placed for decontamination. The testing sensors 16 detect a luminance of the UVC lamp 13, wherein one testing sensor 16 is arranged in each corner of a surface 40 bounded by the dot-dash line. Two further testing sensors 16 are arranged in the center of the surface. The dashed line shown in FIG. 13 corresponds to the irradiation area 23 of the decontamination device 9. A surface 41 of the irradiation area 23 is larger than the surface 40 of the passage 2. The decontamination device 9 comprises indication sensors 17, which are arranged adjacent to the UVC lamp 13.

A method for transferring objects 7 into a controlled environment 3 and a transfer device 1 having a controlled environment 3 comprising a passage 2 are provided, wherein the passage 2 is closable using a cover 5 from the inside 4, in particular inside the controlled environment 3, and is closable from the outside 8 using a package 6 containing an object 7 to be transferred, and having a decontamination device 9, which is configured for a decontamination of the passage 2, wherein a checking unit 10 is configured for a check of a function of the decontamination device 9.

### LIST OF REFERENCE NUMERALS

1 transfer device
2 passage
3 controlled environment
4 inside
5 cover
6 package
7 object
8 outside
9 decontamination device
10 checking unit
11 emission unit
12 H₂O₂ nebulizer
13 UVC lamp
14 sensor
15 testing field
16 testing sensor
17 indication sensor
18 point
19 decontaminating agent
20 detection area of the checking unit
21 surface of the detection area
22 surface of the passage
23 emission area
24 worst-case position
25 checking position
26 operating position
27 access
28 handling device
29 isolator
30 airflow
31 seal
32 housing wall
33 interior of the package
34 inner cover
35 cover
36 container receptacle
37 checking package
38 filling device
39 circumference
40 surface of the passage
41 surface of the irradiation area

1. A transfer device (1), comprising:
a controlled environment (3);
a passage (2), wherein the passage (2) is closable using a cover (5) from inside (4) and is closable from the outside (8) using a package (6) containing an object (7) to be transferred;
a decontamination device (9) configured for a decontamination of the passage (2); and
a checking unit (10) configured for a check of at least one function of the decontamination device (9).

2. The transfer device (1) as claimed in claim 1, wherein the decontamination device (9) comprises at least one of an emission unit (11) or a decontaminating agent distribution device.

3. The transfer device (1) as claimed in claim 2, wherein the emission unit (11) is a radiation generator, and the decontaminating agent distribution device is an H₂O₂ nebulizer (12).

4. The transfer device (1) as claimed in claim 1, wherein the decontamination device (9) is arranged in at least one of the cover (5) or the passage (2).

5. The transfer device (1) as claimed in claim 1, wherein the checking unit (10) is arranged at least one of inside the passage (2) or outside the passage (2).

6. The transfer device (1) as claimed in claim 1, wherein the checking unit (10) comprises at least one sensor (14), and the at least one sensor (14) is arranged at least one of in the decontamination device (9) or outside the decontamination device (9).

7. The transfer device (1) as claimed in claim 6, wherein the at least one sensor (14) comprises at least one of a testing sensor (16) by which the decontamination device (9) is testable, or an indication sensor (17), which triggers a check.

8. The transfer device (1) as claimed in claim 1, wherein the checking unit (10) comprises a checking means sensitive to a decontaminant used on the package (6).

9. The transfer device (1) as claimed in claim 1, the checking unit (10) is arranged in the decontamination device (9).

10. The transfer device (1) as claimed in claim 1, wherein the checking unit (10) and the decontamination device (9) are movable in relation to one another.

11. The transfer device (1) as claimed in claim 10, wherein the checking unit (10) and the decontamination device (9) are movable in relation to one another to change between a checking position (25) and an operating position (26) and/or such that the decontamination device (9) can be brought into or out of a detection area (20) of the checking unit (10).

12. The transfer device (1) as claimed in claim 1, wherein the decontamination device (9) is at least temporarily movable into the controlled environment (3), and/or the checking unit (10) is arranged in the controlled environment (3).

13. The transfer device (1) as claimed in claim 1, wherein the transfer device (1) comprises a comparison unit, which is configured to compare a quantity measured by at least one sensor (14) with a parameter, including at least one of a value range, a setpoint value, a minimum value, or a limiting value.

14. A method for transferring an object (7) into a controlled environment (3), the method comprising:
closing a passage (2) from outside using a package (6) which contains the object (7),
subjecting at least a part of the package (6) to a decontamination using a decontamination device (9) before bringing the object (7) into the controlled environment (3), and
checking a function of the decontamination.

15. The method as claimed in claim 14, further comprising presenting the decontamination device (9) to a testing sensor (16), for example at time intervals and/or after achieving a predetermined criterion.

16. The method as claimed in claim 14, further comprising presenting an emission unit (11), after a release of an access (27) and/or before a closing of the access (27) and/or between two decontamination cycles of the controlled environment (3), to an external sensor (28) to determine a radiation dose.

17. The method as claimed in claim 14, further comprising comparing a quantity measured by the testing sensor (16) and/or an indication sensor (17) with a parameter, including at least one of a value range, a setpoint value and/or a minimum value and/or a limiting value.

18. The method as claimed in claim 17, further comprising a comparison unit outputting an alarm in an event of a deviation of the quantity measured from the parameter and/or notes the deviation in a memory.

19. The method as claimed in claim 14, further comprising presenting the decontamination device (9), during opening of the package (6) and/or removal of contents of the package (6), at least temporarily to the checking unit (10).

20. The method as claimed in claim 14, further comprising comparing a measurement result of at least one sensor (14), which is arranged in the decontamination device (9), with a measurement result of a sensor, which is arranged outside the decontamination device (9).
